(19) United States
(12) Patent Application Publication
(10) Pub. No.: US 2021/0282517 A1
(43) Pub. Date: Sep. 16, 2021

(54) Germ Fighting Reserve System

(71) Applicant: Gary Trenda, Valencia, CA (US)

(72) Inventor: Gary Trenda, Valencia, CA (US)

(21) Appl. No.: 17/199,743

(22) Filed: Mar. 12, 2021

Related U.S. Application Data
(60) Provisional application No. 62/988,615, filed on Mar. 12, 2020.

(51) Int. Cl. A45C 11/00; B65D 43/16; B65D 43/22; B65D 25/04; A45F 3/04; A45F 3/00; A45C 13/02; A61L 2/26; A61L 9/014; A61L 9/14; A61L 2/22; A61L 2/00

(52) U.S. Cl. CPC A45C 11/00; A61L 2209/22; B65D 43/22; B65D 25/04; A45F 3/04; A45F 3/005; A45C 13/02; A61L 2/26; A61L 9/014; A61L 9/14; A61L 2/22; A61L 2/0088; A45C 2011/007; A45F 2003/003; A61L 2202/16; B65D 43/163

(57) ABSTRACT

A virus and germ fighting system. The system includes a main container having a base, sidewalls, and an upper opening defining an interior volume. The interior volume includes a plurality of divided compartments. Each compartment may include its own locking lid. The compartments include specific virus and germ fighting materials. A first compartment includes replaceable disinfectant spray containers, a second compartment includes travel containers and other portable germ fighting items, and the third compartment includes a full body suit for enhanced protection when needed. The system also includes a user device that is in wireless communication with a database server via a network. The user device can be used to resupply the system's components via communication with the database. The database is configured to automatically provide updated information regarding instructions for how to properly and effectively combine the components of the system to prevent the spread of germs and viruses.

### CROSS REFERENCE TO RELATED APPLICATIONS

This application claims the benefit of U.S. Provisional Application No. 62/988,615 filed on Mar. 12, 2020. The above identified patent application is herein incorporated by reference in its entirety to provide continuity of disclosure.

### BACKGROUND OF THE INVENTION

The present invention relates to systems for preventing the spread of germs and viruses. More specifically, the present invention provides a comprehensive system that includes an organized container with multiple virus and germ fighting items, wherein the system further provides users with the knowledge to properly disinfect their surroundings, whether in their own home or in a more public environment.

Average households and businesses lack an organized system and the necessary protective devices to fight new germ and flu invasions into their home or businesses. Scientific studies indicate that 5% to 20% of the U.S. population gets the flu every year. There has been a rise to 58% of the deaths due to flus and viruses of adults of age over 65. The old concept that senior citizens are the only people at higher risk is no longer valid. Now we have learned that children younger than the age of two to five, pregnant women, people with weakened immune system, people with chronic illnesses and people who are obese are now added to the list of those in danger.

The world was not prepared for the recent pandemic and COVID-19 virus. The recent pandemic proved that almost every household failed to have basic pandemic supplies that were absolutely needed to fight Covid-19. Huge shortages of disinfectants, mask, gloves, hand soap, and gowns occurred immediately. Many people seeking these items were met with supply shortages and price gouging. If anything is to be learned from this is that everyone including our government needs to have stored a pandemic supplies system. Germs and viruses are constantly changing, and it is becoming more and more difficult to find cures. It is now imperative to have a ready and available system in your home and place of business because a small delay can become a serious illness. Germs are also lasting longer on surfaces and in the air and are becoming faster and quicker in transferring from one surface to another. Humans becoming infected with a serious illness is unfortunately happening easier and faster than ever before.

The average household or business usually has little to no supplies stored or organized in place when the first signs of a problem occur. When someone becomes ill, they often use medicine that is found in their medicine cabinet and wait for results, hoping the illness goes away. That medicine is usually old and has lost its effectiveness or may be a medicine that has little or no effect on the germs they are fighting. When that fails, the next step is usually to go to the store and look for anything they think might help. Individuals are often unaware of the exact items they may need to help. Pandemics and other emergencies can cause shortages that may deny the individual the supplies they desperately need. This delay leaves them venerable to serious infections. The final step is typically seeing a doctor. Meanwhile this lack of protective procedures allows the germs to infect not only the person that was first infected but also these germs now have the opportunity to attack other members of the family or business associates. This form of delay has now become a serious problem.

Individual devices exist that can be utilized to combat disease and germ spread. Gloves, hand sanitizer, disinfecting sprays, and masks can all be utilized independently to fight germs. The average household does not have the protective devices available to them at the time they need it most. Also, it is typically the case that one specific device will only partially prevent the spread of disease and germs. It can be difficult to gather, store, and transport enough independent devices to really make a difference in air quality or the amount of germs and diseases on a particular surface. In view of these concerns and more, it is desirable to provide a virus and germ fighting system that encloses each of these individual devices within a single, accessible interior, as well as providing information for how to most effectively use the system.

In light of the devices disclosed in the known art, it is submitted that the present invention substantially diverges in design elements from the prior art and consequently it is clear that there is a need in the art for an improvement to existing knee pad devices. In this regard the present invention substantially fulfills these needs.

### SUMMARY OF THE INVENTION

In view of the foregoing disadvantages inherent in the known types of knee pad devices now present in the prior art, the present invention provides a virus and germ fighting system wherein the same can be utilized to prevent the spread of viruses and germs in private and public environments.

As a general overview, the system includes a main container having a base, a plurality of sidewalls, and an upper opening defining an interior volume. A lid is pivotally attached to one of the sidewalls. A locking latch is affixed to the lid configured to engage a locking receiver affixed to one of the sidewalls. A plurality of interior compartments are disposed within the interior volume. A plurality of disinfectant containers are removably secured within a first interior compartment. A travel bag is removably secured within a second interior compartment, the travel bag including a plurality of germ fighting items. A sealable body suit is removably secured within a third interior compartment. The virus and germ fighting system further includes a user device, such as a smartphone or other computer, in wireless communication with a database, wherein the user device is configured to receive information regarding correct usage of the plurality of germ fighting items, and wherein the database is configured to receive and carry out resupply requests.

A primary object of the present invention is to provide a pandemic reverse system which fights germs daily and can provide additional protection in emergency and pandemic situations. By being prepared with the present pandemic reserve system, individuals can prevent the spread of germs and diseases proactively. Rather than having to purchase products and gather information after a pandemic occurs, the present invention provides all of the necessary tools and information to protect oneself before a pandemic outbreak begins, or immediately after one is detected.

Another main object of the present invention is to provide a system that is prepared for when someone gets sick. The timing of preventative measures and treatment is crucial. The present system allows germs to be prevented from spreading and further allows for immediate response when someone becomes ill. Rather than the ill person having to search their medicine cabinet, go to the store, or the like, the present system provides them with the tools and knowledge to immediately take action to prevent germs from spreading and combating disease.

One object of the present invention is to provides a virus and germ fighting system that utilizes multiple virus and germ fighting tools and keeps them stored in an easily accessible manner, as well as provides for the ability to automatically restock on supplies.

Another object of the present invention is to provide a virus and germ fighting system that also provides knowledge of the right equipment, the right products, the appropriate disinfectants, and the appropriate time to use the individual tools of the system.

A further object of the present invention is to provide a virus and germ fighting system that includes sterilized protective equipment, such as masks, gloves, and shields, for example, with safety features and instructions to prevent improper use of such equipment by the user.

Yet another object of the present invention is to provide a virus and germ fighting system that is focused on preventing infections and illnesses from occurring in the first place.

Still a further object of the present invention is to provide a virus and germ fighting system that includes all necessary germ and disease fighting chemicals and tools in one or more easily accessible containers, including containers that are specifically designed for use during travel or in public spaces where environmental control is limited.

Another object of the present invention is to provide a virus and germ fighting system that creates a disconnection along the line of germ transfer by providing the ability to take immediate action to defend oneself from, flus and viruses as soon as possible in public places, as well as creating a safe space in your home or business.

Yet another object of the present invention is to provide a virus and germ fighting system that includes a container that stores disinfectants in a safe locked container or a cardboard box flap and tie system.

A further object of the present invention is to provide a virus and germ fighting system that provides updated information regarding new germ fighting techniques and products, such that the system can be adaptable to improved methods and devices as they are developed and tested over time.

Other objects, features, and advantages of the present invention will become apparent given the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the virus and germ fighting system showing the main container.

FIG. 2 shows a perspective view of an embodiment of the virus and germ fighting system showing one of the compartments that are within the main container.

FIG. 3 shows a perspective view of an embodiment of the virus and germ fighting system having additional storage within the lid.

FIG. 4 shows a perspective view of an embodiment of the virus and germ fighting system showing a backpack embodiment of the travel bag that is stored within the main container.

FIG. 5 shows a perspective view of an embodiment of the virus and germ fighting system showing a fanny pack embodiment of the travel bag that is stored within the main container.

### DETAILED DESCRIPTION OF THE INVENTION

Reference is made herein to the attached drawings. Like reference numerals are used throughout the drawings to depict like or similar elements of the virus and germ fighting system. For the purposes of presenting a brief and clear description of the present invention, the preferred embodiment will be discussed as used for providing a system with multiple germ and virus fighting tools for public and private use, as well as information for how to best use the system and its individual components. The figures are intended for representative purposes only and should not be considered to be limiting in any respect.

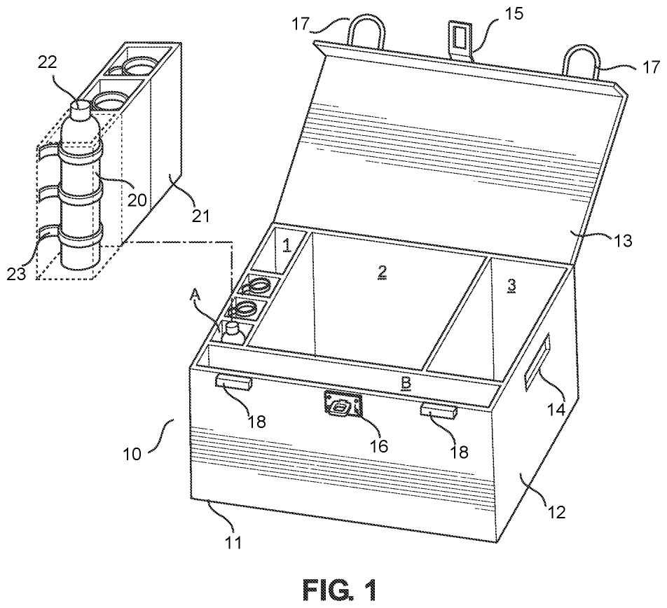

Referring now to FIG. 1, there is shown a perspective view of an embodiment of the virus and germ fighting system showing the main container. The main container 10 includes a base 11, a plurality of sidewalls 12, and an upper opening defining an interior volume that is subdivided into three interior compartments 1, 2, and 3. A lid 13 is pivotally attached to one of the sidewalls 12. A locking latch 15 is affixed to the lid, which is configured to engage a locking receiver affixed to one of the sidewalls 12. In the shown embodiment, the lid can be divided into three separate sections, such that the side sections have individual locking latches and receivers 17, 18. This allows the user to lock the compartments 1, 2, and 3 and access one at a time as needed, keeping the contents of the remaining compartments safe and secure until needed. In other embodiments, other types of locking closures may be utilized, and any suitable type of lock may be utilized. In the shown embodiment, the sidewalls also include handles such as cutouts 14 that assist the user with transporting the main container 10 if needed.

The plurality of interior compartments 1, 2, 3 are configured for specific uses. The first compartment 1 is adapted to receive a plurality of disinfectant containers 20. The containers 20 are secured within a secure storage mechanism that may be fixed within the compartment or removably secured within the first interior compartment. Each disinfectant container 20 has a special holding system that includes adjustable retaining rings 23 that connect to interior portions of the individual container supports 21 that are divided within the first compartment 1. As shown, the disinfectant containers include caps, but some may include different types of spray nozzles. Further, the shape and size of the containers 20 can vary. For example a commercial version would include larger containers 20 and different types of sprayers or other accessories compared to a personal or in-home version which would have smaller containers 20. The retaining rings 23 are designed to avoid spills commonly found when disinfectants are stored under the sink, and each holder is designed to make it difficult for a child to pull out the container 20. In operation, a user may order the compartment with refilled containers 20 as they reorder using the system's internet capabilities. Also, users can utilize the present system with their own safety containers if they wish to do so.

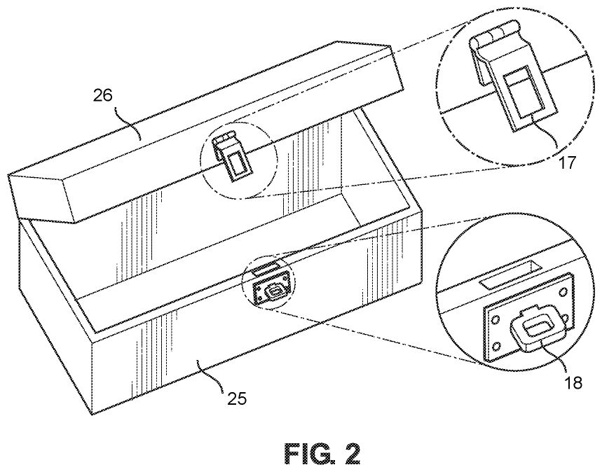

As shown in FIG. 1, the container can have additional empty sections that can be customized for different item storage. For example, if using the germ fighting system in a larger public space, the user may need additional masks or gloves, or a larger supply of stronger disinfectants. A household might need four different sizes pandemic full body suits. One can build a better reserve system for their business and home by being able to purchase these specialty items over a period of time using the present system. The system further automatically updates the user, through communication between a user device such as a smartphone or other computer and a database, regarding use instructions and other important information. The system can further notify users based on date ranges when new items should be ordered. In some embodiments, the individual compartments are entirely removable, as shown in FIG. 2, so the user can replace them with new compartments having new items when needed.

The first compartment of the system houses items that are specifically designed for preventing germs from entering the body while at home, at work, or in other semi-confined, semi-controllable environments. These may include, but are not limited to: a mask, a pair of gloves, replacement mask filters, goggles, portable germ barrier walls, disinfectants, ear protectors, and thermometers. Any number and combination of the above may be included, as well as other germ prevention items that would assist in the overall goal of preventing disease transmissions, as the system is designed to be automatically updated over time as new information and techniques become available.

Regarding the glove, the glove is preferably that disclosed in U.S. patent application Ser. Nos. 16/186,968 and 17/031,409, which are directed to a “reusable hand article for cleaning”. It is important to provide this cleaning glove to replace ineffective disposable wipes, disposable gloves, germ-harboring rags, and other ineffective cleaning tools. The system preferably includes a first glove designed for initial surface cleaning along with the disinfectants provided by the system. The second glove is designed to spread the disinfectant to increase effectiveness. The gloves prevent chemical contact with the user's hand while allowing for a tactile feel to determine exactly what areas have been cleaned.

Regarding masks, individuals often wear improper masks, or wear proper masks improperly. The system's database explains, via application or website, how to correctly use a proper mask based on up-to-date scientific information. The system includes a combination of different masks for different purposes or environments. The system includes a surgical mask, a respirator and air mask, charcoal filter mask, and a reusable mask (which can replace charcoal filters and be disinfected). Each mask is labeled and is designed for specific task. Further, each mask and reusable filter includes date markers or the like for easily monitoring usage and determining when to change masks or filters. The database, via the website or user application, provides the knowledge to the user required to maintain a mask performance of protection. For example, the instructions will outline on how to fit the mask, how to clean and disinfect it and how to know when to change the mask and filters. The instructions also show how to store the mask when not in use to give the mask a longer life.

Regarding goggles, the system can include goggles to protect the eyes. The goggles may include UV protection, infrared or night vision properties, and other accessories that increase their usability. The goggles may also be an optional feature.

The germ barrier wall allows for isolation of a user in an environment in which they typically would not have control or be able to isolate. As one example, the germ barrier wall can be used to isolate from a sick coworker, or isolate on public transport. Isolation helps prevent spread of germs. There are several kinds of barriers the system includes, such as acrylic germ barriers, sneeze guards, room dividers which main purpose to keep people safe and reduce the risk of infection. The user can customize their order via the website, application, and database to include specific types of germ barriers.

Section one also contains the mask needed to shorten exposure from strong disinfectants that may contain phthalaldeyde, hydrogen peroxide and/or peracetic acid. The website and database provides information on how to properly use the disinfectants, including the proper two-step cleaning process using both a cleaning liquid and a disinfectant. The system can be customized by the user to have different types and amounts of disinfectants. Each container includes precise labeling and color coding for easy identification and selection. Each container also includes a solid line indicator showing liquid level to remind of reordering. In some embodiments, the system automatically transmits to the user device a reminder to reorder based on expiration dates or order dates that are stored in the database.

The first section can include additional features such as ear plugs or ear coverings that prevent germs from entering through the ears. It may also include a thermometer having an infrared temperature sensor, or a wearable wrist thermometer. As with the rest of the system, the individual components of the second system can be customized by the user, and instructions on how to use particular combinations of items effectively will be provided to the user.

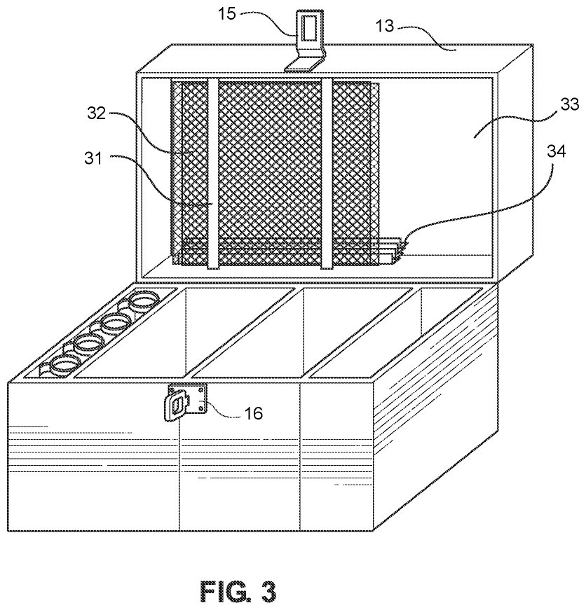

Referring now to FIG. 3, there is shown a perspective view of an embodiment of the virus and germ fighting system having additional storage within the lid. In some embodiments, the lid 13 includes an interior storage area 33 with retaining straps 31. The storage area 33 can be configured to store travel air filters, such as those covered in U.S. patent application Ser. No. 16/009,438, directed to a portable air filter device and method. With this embodiment, the storage are 33 is configured to store the adjustable filter support frames 34 and multiple replacement filters 32. The filter frames 34 can be adjusted to secure the filters 32 over different types and sizes of airflow ducts in order to filter air from germs and other contaminants. For example, the filters 32 can be secured over an air duct in a hotel room or a vent in a public transport vehicle for added protection.

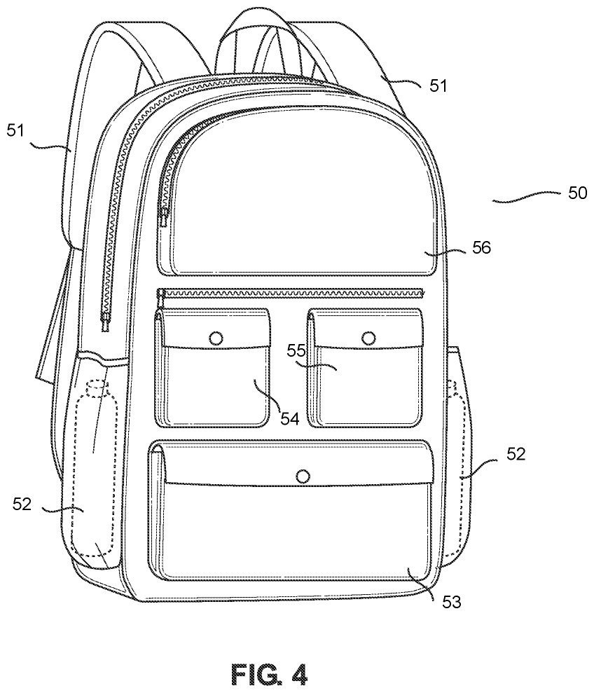

Referring now to FIG. 4, there is shown a perspective view of an embodiment of the virus and germ fighting system showing a backpack embodiment of the travel bag that is stored within the main container. The second compartment of the container includes different travel containers for using the system while traveling or otherwise away from home. The travel containers are preloaded with different germ fighting items. For example, as shown in FIG. 4, the travel container can be a backpack 50 with shoulder straps 51. The backpack can include side pockets 52 that are preloaded with disinfectant sprays, sanitizers, or other liquids. The remaining compartments of the backpack 53, 54, 55, 56 can be individually prefilled with other items necessary for germ fighting on the go.

In one embodiment, the backpack is specially designed to store 16 to 32 oz bottle of disinfectants, 2 to 4 pairs of reusable gloves, a respirator mask, a set of 2-4 latex gloves, a package of surgical gloves, 2 carbon mask filters, a carbon hat, goggles, travel filters and air spray disinfectants, doorknob covers with warning labels such as ‘do not enter’, and steering wheel covers, for example. In some embodiments, the material used to construct the backpack creates a soft-sided suitcase and has on the outside loops.

The backpack embodiment of the travel bag is intended for use on longer trips, such as overnight trips and the like. The sizes and number of items are tailored for this purpose. The backpack provides a person the items necessary to have in their possession when traveling outside their home or office for a long period of time. This backpack is ideally designed for overnight weekend trips and a such provides specific protective items. For example, the items in the backpack will allow users to disinfect and secure a hotel room or other space that they must occupy, over which they traditionally would have no environmental control.

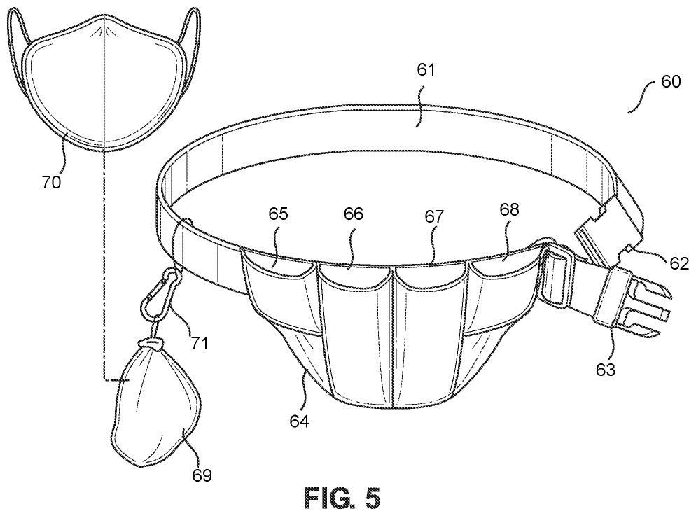

Referring now to FIG. 5, there is shown a perspective view of an embodiment of the virus and germ fighting system showing a fanny pack embodiment of the travel bag that is stored within the main container. The fanny pack 60 includes a waist strap 61 that is adjustable via a slide buckle and receiver mechanism 62, 63. The front pouch 64 includes individual pockets 65, 66, 67, and 68 that house specific germ fighting items. A pouch 69 is connected to the waist strap 61 via a clip 71. The pouch 69 stores a facemask 70 therein, such that it is easily accessible for the user.

The fanny pack embodiment of the travel bag is designed to provide the necessary disinfecting and germ fighting tools for short trips, such as to and from the grocery store, as one example. The liquid containers are sized to be compliant with federal aviation rules, such that the user can easily take the fanny pack on a plane. The items in the fanny pack, such as disinfecting wipes, masks, filters, gloves, and the like, can be utilized to disinfect, sanitize, and secure the immediate area around the user as they sit on a plane, bus, or other public space. Thus, the fanny pack is specifically designed for shorter outings, whereas the backpack is designed for longer outings or overnight trips.

The second section is designed to provide the user protection when the user leaves your home. This could be as simple as shopping or taking a long vacation. In one embodiment, the compartments of the germ fighting fanny pack are designed to specifically carry two three oz. bottles for disinfectants and inside the pouch contain the aforementioned reusable hand article for cleaning, a latex glove, a surgical mask, and a carbon mask. This fanny pack is made for the purpose of having a defensive approach to contacting germs on all your daily trips.

The third section of the system includes a full body suit that can be sealed from the external environment to isolate the user from all germs. The full body suit can include a transparent hood for vision. The full body suit sizes and shapes can vary but are included as an emergency protective measure in the system. This section also includes a respirator such as an N95 or higher rated mask. In highly contagious pandemics or other emergencies, it may be desirable to utilize such increased protection.

The present system can include additional components for fighting germs and viruses. The user can customize the exact number and types of components in there system along with help from the information provided by the system's database. For example, the system can also include steamers, air purifiers, cell phone cleaners, spray mops for floor cleaning, UV sanitizers, and any other desirable device that helps further the goal of reducing the spread of germs and contraction of diseases. As another example, the system can include toilet seat covers, vomit gags, hand sanitizers, face shields and plastic covers, and a bubble enclosure including a shoulder-mounted umbrella-like bubble that prevents the spread of viral particles via moisture droplets from the nose and mouth. As yet another example, the system can include a face mask and shield assembly that includes an airflow tube that forces air up away from the upper end of the mask, providing a fresh air flow for the user and preventing germ and viral spread thereby.

The virus and germ fighting system constantly updates the information of potential pandemic warning to provide potential time to prepare. The database also gives updates on availability of products one might need to have in order to prepare for a biological attack. The information provided is designed to provide necessary help and assistance in providing the consumer information and services they need to help prevent existing as well as new germs and flus that infect the body and spreading throughout their space. For example, the database provides knowledge of what disinfectants to use, where to use them, how to use them safely, how often and where to use them. The database can be updated overtime with new instructions and new information as science evolves and new products emerge to further the disease prevention goal. The database can communicate via an application and website which provide a quick and easy communication when the user needs it most, especially when preventing germs entering the user's space or when someone in the user's space is ill. In either case time, is of the essence, and these preventative measures must be taken quickly and effectively. The present invention therefore provides most if not all the products needed to immediately fight the invasion of germs into public and private spaces.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A virus and germ fighting system, comprising:
a main container having a base, a plurality of sidewalls, and an upper opening defining an interior volume;
a lid pivotally attached to one of the sidewalls;
a locking latch affixed to the lid configured to engage a locking receiver affixed to one of the sidewalls;
a plurality of interior compartments disposed within the interior volume;
a plurality of disinfectant containers removably secured within a first interior compartment;
a travel bag removably secured within a second interior compartment, the travel bag comprising a plurality of germ fighting items;
a sealable body suit removably secured within a third interior compartment.

2) The virus and germ fighting system of claim 1, further comprising a user device in wireless communication with a database, wherein the user device is configured to receive information regarding correct usage of the plurality of germ fighting items.

3) The virus and germ fighting system of claim 1, wherein the first interior compartment is removable from the main container.

4) The virus and germ fighting system of claim 1, wherein the disinfectant containers are secured within the first interior compartment via a plurality of flexible retaining rings disposed within the first interior compartment.

5) The virus and germ fighting system of claim 2, wherein the database is configured to receive resupply requests from the user device.

6) The virus and germ fighting system of claim 1, wherein the travel bag is a backpack having a pair of shoulder straps.

7) The virus and germ fighting system of claim 1, wherein the travel bag is a fanny pack having a waist strap.

8) The virus and germ fighting system of claim 1, wherein the travel bag includes multiple pockets preloaded with a plurality of germ fighting items.

9) The virus and germ fighting system of claim 1, wherein each compartment of the plurality of compartments includes its own locking mechanism, such that each compartment of the plurality of compartments can be locked or accessed individually.

10) The virus and germ fighting system of claim 1, further comprising a lid storage area, the lid storage area supporting a plurality of adjustable filter frames and a plurality of filters.

11) The virus and germ fighting system of claim 1, further comprising a pair of retaining straps extending across the lid storage area configured to secure the adjustable filter frames and the filters therein.

12) The virus and germ fighting system of claim 1, wherein the database is configured to transmit information regarding reorder dates based on an amount of time having elapsed after a prior restock of items in the main container.

13) The virus and germ fighting system of claim 1, wherein the plurality of germ fighting items are selected from the group of: a glove, a mask, a disinfectant spray, a pair of goggles, a face shield, a shoulder mounted bubble enclosure, a toilet seat cover, a vomit bag, and a hand sanitizer.
